(19) United States
(12) Patent Application Publication
(10) Pub. No.: US 2021/0361671 A1
(43) Pub. Date: Nov. 25, 2021

(54) COMBINATION THERAPY CONTAINING IMMUNE BOOSTERS, DIGESTIVE ENZYMES AND INTERFERONS FOR CANCER TREATMENT

(71) Applicant: Ulrich Jentschura, Rolla, MO (US)

(72) Inventor: Ulrich Jentschura, Rolla, MO (US)

(21) Appl. No.: 16/758,735

(22) PCT Filed: Oct. 25, 2018
(86) PCT No.: PCT/US18/57454

§ 371 Date: Apr. 23, 2020

Related U.S. Application Data
(60) Provisional application No. 62/577,284, filed on Oct. 26, 2017.

(51) Int. Cl. A61K 31/56; A61K 36/81; A61K 31/375; A61K 38/21; A61K 38/48; A61K 31/198; A61K 36/28; A61K 31/07; A61K 31/4415; A61K 31/714

(52) U.S. Cl. CPC A61K 31/56; A61K 36/81; A61K 31/375; A61K 38/21; A61K 38/4886; C12Y 304/2404; A61K 36/28; A61K 31/07; A61K 31/4415; A61K 31/714; A61K 31/198

(57) ABSTRACT

Methods and compositions for treating cancer in a patient in need thereof are provided. The methods comprise administering an interferon or an extract containing the interferon, an immune promoting agent and a digestive enzyme to the patient. The interferon can comprise withaferin A; the immune promoting agent can comprise vitamins and the digestive enzyme can comprise serratiopeptidase.

### REFERENCE TO PREVIOUSLY FILED APPLICATION

This application claims benefit of U.S. provisional application Ser. No. 62/577,284, filed Oct. 26, 2017, the entire disclosure of which is incorporated herein by reference.

### FIELD OF THE INVENTION

The present disclosure generally relates to methods for treating a malignancy, a pre-malignant condition or cancer in a subject in need thereof. The methods comprise administering to the subject an interferon or an extract containing the interferon, an immune promoting agent and a digestive enzyme.

### BACKGROUND OF THE INVENTION

Malignant, pre-malignant and cancerous conditions all encompass various diseases characterized by abnormal cell growth. While excessive cell growth can exist and form benign, localized tumors, a malignant condition or cancer is characterized by the ability to metastasize and invade other organs. Cellular changes that result in malignancy are also usually detectable in “pre-malignant” conditions, which must be observed or treated aggressively to prevent emergence of a malignant tumor. These conditions are caused by a variety of intrinsic and extrinsic factors and is treated differently depending on the initial site of growth and other factors.

Common treatments for a malignant or pre-malignant condition or cancer include surgery (resection), chemotherapy, and radiation therapy. These treatments are usually indiscriminant and cause local and systemic tissue damage and severe side effects. Moreover, a significant population of patients are unresponsive to these traditional therapies or are poor candidates for them (e.g. for inoperable tumors). Therefore, there is a need for new cancer treatments having less severe effects that can help these nonresponsive patient populations as well as treat responsive patient populations.

Immunotherapy has emerged recently has a novel cancer treatment and functions by harnessing the body's immune system to fight cancer. Immunotherapies, which can be categorized as active, passive or hybrid, exploit the fact that cancer cells express and display surface molecules, referred to as tumor-associated antigens (TAAs), which can be detected and targeted by the immune system. Active immunotherapy directs the immune system to attack tumor cells by targeting specific TAAs while passive immunotherapies enhance existing anti-tumor responses. However, while promising, many immunotherapies are also plagued by severe side effects since they trigger a massive immune response that can be difficult to localize to the tumor or cancer to be treated.

Accordingly, there is an ongoing need for methods that can effectively treat a malignancy, a pre-malignant condition or cancer without causing detrimental side effects that can reduce the satisfaction of life for the patient.

### SUMMARY OF THE INVENTION

A method for treating a malignancy, a pre-malignant condition or cancer is provided, the method comprising administering an interferon or an extract containing the interferon, an immune promoting agent, and a digestive enzyme to a subject in need thereof. The use of a composition as a medicament is also provided to treat a malignancy, a pre-malignant condition or cancer in a subject in need thereof, the composition comprising an interferon or an extract containing the interferon, an immune promoting agent, and a digestive enzyme.

Other objects and features will be in part apparent and in part pointed out hereinafter.

### DETAILED DESCRIPTION OF THE INVENTION

It has been discovered that a unique combination of an interferon or an extract containing the interferon, an immune promoting agent and a digestive enzyme is beneficial and useful for treating a malignant, pre-malignant or cancerous condition. The therapy discovered has found to have a number of advantages over current treatment regimes. For example, the methods and compositions described herein offer improved options for early treatment (e.g., when a pre-malignant condition is observed rather than actively and aggressively treated immediately). In addition, the methods described herein offer improved outcomes when used with traditional treatment and improve the immune response to cancer cells. Ultimately, when administered according to the methods described herein, the interferon or extract containing the interferon, an immune promoting agent and digestive enzyme can lead to improved survival rates and reduced treatment costs.

### 1. Methods

A method of treating a malignancy, a pre-malignant condition, or cancer in a subject in need thereof is provided, the method comprising administering to the subject an interferon or an extract containing the interferon, an immune promoting agent and a digestive enzyme.

Also provided is a use for a composition as a medicament to treat cancer in a subject in need thereof, the composition comprising an interferon or an extract containing the interferon, an immune promoting agent and a digestive enzyme.

The interferon administered to the subject is a substance capable of promoting cytotoxicity. The interferon can be naturally or synthetically derived. Preferably, the interferon is derived from the Ashwaghanda extract taken from the Withaferania somnia plant. The extract can be taken from any part of the plant (i.e. leaves, stem, roots, or bark) and can be obtained using any method known in the art. Preferably, the Ashwaghanda extract comprises the roots of the plant. Preferably, the extract containing the interferon can comprise the Ashwaghanda extract. Preferably, the interferon or extract containing the interferon comprises a withanolide (e.g., withaferin A).

The extract containing the interferon can be administered at a dose of about 500 mg to about 2000 mg, or from about 600 mg to about 1800 mg, or from about 800 mg to about 1600 mg, or from about 1000 mg to about 1400 mg a day. Preferably, the extract containing the interferon can be administered at a dose of about 1100 to 1300 mg a day.

The interferon can also be isolated and/or purified from the extract. Alternatively, a synthetic interferon could be used. Therefore, the methods described herein can comprise administering the interferon (e.g., in lieu of the extract). When the interferon is administered in lieu of the extract, the dose can be determined from the amount of interferon in the appropriate dose of extract to be administered. For example, the dose of interferon to be administered can correspond to the amount of interferon present in an appropriate dose of the extract containing the interferon. For example, when the interferon comprises withaferin A and the extract containing the interferon comprises the Ashwaghanda extract, then the interferon (e.g., withaferin A) can be administered as a dose of the Ashwaghanda extract (using the doses listed above) or alone at a dose that corresponds to the amount of interferon (e.g., withaferin A) present in the appropriate dose of the Ashwaghanda extract.

When the interferon comprises withaferin A (or another interferon having a high metabolic rate), the interferon or extract containing the interferon can be administered multiple times a day. For example, the interferon or extract containing the interferon can be administered 4 times a day to reach the maximum daily dose as described above.

Daily administration of any of the components described herein can comprise a single bolus dose or can comprise multiple doses that collectively result in the allotted daily dose.

The immune promoting agent as used in the method is any substance that elicits or promotes an immune response. Vitamins are particularly useful immune promoting agents. Therefore, the immune promoting agent can comprise vitamin C (ascorbic acid), vitamin A (beta-carotene or retinol), Vitamin B₆ (pyridoxine), Vitamin B₁₂ (cyanocobalamin) or any combination thereof. In some cases, vitamin B₆ and B₁₂ can be administered in a complex (e.g., vitamin B₆-B₁₂ complex).

The immune promoting agent can be administered at the recommended daily allowance (RDA) for each component. For example, vitamins A, B₆ and B₁₂ can be administered according to the recommended daily allowance (RDA) for each substance. Alternatively, the immune promoting agent can be administered at a concentration much greater than the recommended daily allowance (RDA). The immune promoting agent can be administered at a rate 10 to 50 times greater, 20 to 40 times greater, 30 to 40 times greater, or 30 to 35 times greater than the RDA for the agent. For example, the RDA for vitamin C is around 75 mg to 90 mg. Therefore when the immune promoting agent comprises vitamin C, the agent can be administered at a dose of about 1000 mg to about 5000 mg, 2000 mg to about 4000 mg, or 2500 mg to about 3500 mg a day. Vitamin C can also be administered at a dose of at least about 1000 mg, at least about 2000 mg or at least about 3000 mg a day. For example, Vitamin C can be administered at a dose of at least 2000 mg or about 3000 mg a day.

The digestive enzyme of the method can comprise any enzyme capable of degrading cellular tissues. For example, the digestive enzyme can comprise a proteolytic enzyme. The proteolytic enzyme can be obtained, for example, from the enterobacterium Serratia sp. E-15. Preferably, the digestive enzyme comprises serratiopeptidase or serrapeptase.

The digestive enzyme can be administered at a daily dose of about 300,000 to about 400,000 active units, from about 310,000 to about 390,000 active units, or from about 330,000 to about 370,000 active units. Preferably, the enzyme is administered at a dose of about 360,000 active units per day.

Without being bound by theory, it is believed that the combination therapy of the interferon, immune promoting agent and digestive enzyme acts synergistically to treat cancer. Specifically, the immune promoting agent primes the immune system to recognize and attack cancerous cells; the interferon suppresses cell growth and the digestive enzymes facilitate the degradation and removal of the dead cells. It is believed that when the rate of cell elimination exceeds the growth rate of the tumor, the treatment can result in remission and recovery.

The method can further comprise administering additional herbal supplements that can augment the anti-tumor properties of the combination described above. Suitable herbal supplements can comprise those that possess anti-cancer, anti-inflammatory, or immunomodulatory properties. For example, the herbal supplement can comprise Echinacea. Alternatively, or in addition, the supplement can comprise kachnar (Bauhinia tormentosa) bark extract.

Preferably the additional herbal supplement is administered at an amount of about 100 mg to about 1000 mg, from about 200 mg to about 900 mg, from about 300 mg to about 800 mg, from about 400 mg to about 700 mg, or from about 500 mg to about 700 mg per day. Preferably, the herbal supplement is administered at an amount of about 600 mg per day.

When more than one herbal supplement is administered (e.g., kachnar and Echinacea), each supplement can be administered at an amount of about 100 mg to about 1000 mg, from about 200 mg to about 900 mg, from about 300 mg to about 800 mg, from about 400 mg to about 700 mg, or from about 500 mg to about 700 mg per day. For example, each supplement can be administered at an amount of about 600 mg per day.

### 2. Methods of Administration

The agents and compositions described herein (e.g., interferon or an extract containing the interferon, immune promoting agent, digestive enzyme, herbal supplements) can be administered according to methods described herein by a variety of means known in the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

The agents and compositions can be administered using parenteral, pulmonary, oral, topical, intradermal, intramuscular, intraperitoneal, intravenous, subcutaneous, intranasal, epidural, ophthalmic, buccal, or rectal administration. Preferably, the agents and compositions are administered orally.

Agents and compositions described herein can be administered in a variety of methods well known in the art. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance of the agent; decrease the nonspecific delivery of the agent to non-target tissues; decrease irritation caused by the agent; decrease toxicity due to high initial doses of the agent; alter the immunogenicity of the agent; decrease dosage frequency, improve taste of the product; or improve shelf life of the product.

As noted above, the agents and compositions to be administered can preferably be provided at the doses described above. The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

An advantage of the treatment described herein is the relative low toxicity of the individual components and the relatively benign consequences of an overdose. For example, an overdose of vitamin C typically leads to digestive irregularities; a prolonged overdose of serratiopeptase affects liver function, leading to yellowish discoloring of the stool; and an overdose of Ashwaghanda has been observed to stimulate the thyroid, possibly leading to (benign) goiter growth. However, it is considered that a person of ordinary skill may find it necessary to further optimize the doses described above depending on the type and severity of the malignancy, pre-malignant condition or cancer to be treated. In doing so, the toxicity and therapeutic efficacy of the agents and compositions described herein can be considered.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Shamel (2004) Applied Biopharmaceutics & Pharmacokinetics, McGraw-Hill/Appleton & Lange, ISBN 0071375503). For example, it is well within the skill of the art to start doses of the composition at levels less than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure can be determined by an attending physician within the scope of sound medical judgment.

Again, the advantage of the methods described herein is in the treatment of a malignancy, pre-malignant condition or cancer. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of the agents and compositions described herein can occur as a single event or over a time course of treatment. For example, the interferon/immune promoting agent/digestive enzyme can be administered daily, weekly, bi-weekly or monthly. Preferably, however, the treatment comprising the interferon, immune promoting agent and digestive enzyme is administered daily. For treatment of the conditions as described herein, the time course of treatment will usually be at least several months. For example, treatment could extend over one month, two months, or three months. Generally, for more chronic conditions, treatment could extend from several months to a year or more. In some cases, treatment could extend for more than one year (e.g., one, two or three years). Preferably, the treatment extends over an 18 to 24 month period (e.g., 20 months).

Moreover, the compounds and agents described herein (e.g., the interferon or an extract containing the interferon, immune promoting agent and digestive enzyme) can be administered simultaneously or sequentially. Simultaneous administration can occur through administration of separate compositions, each containing one or more of the compounds as described herein (e.g., the interferon or an extract containing the interferon, immune promoting agent and digestive enzyme). Simultaneous administration can occur through administration of one composition containing two or more of the compounds as described herein. Alternatively, the compounds described herein can be administered sequentially. For example, one or more of the compound as described herein (e.g., the interferon or the extract containing the interferon, immune promoting agent or digestive enzyme) can be administered before or after administration of the other required component (e.g., interferon or the extract containing the interferon, immune promoting agent, or digestive enzyme).

In addition, the agents and compositions described herein can be formulated into suitable pharmaceutical compositions or formulations to optimize their administration.

### 2. Pharmaceutical Compositions or Formulations

The agents and compositions to be administered can be prepared in any number of pharmaceutical compositions or formulations as known in the art to optimize their delivery. Therefore, the agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers. Pharmaceutically acceptable excipients for use in the compositions of the present invention are selected based upon a number of factors including the particular compound used, and its concentration, stability and intended bioavailability; the subject, its age, size and general condition; and the route of administration.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. Routes of administration include, but are not limited to, oral, parenteral (e.g., intravenous, intra-arterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration. For example, the agents of use with the current disclosure can be formulated by known methods for administration to a subject using several routes including: parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, van der Waals, hydrophobic, hydrophilic or other physical forces.

The pharmaceutical compositions can be formulated, for example, for oral administration. The pharmaceutical compositions can be formulated as tablets, dispersible powders, pills, capsules, gel-caps, granules, solutions, suspensions, emulsions, syrups, elixirs, troches, lozenges, or any other dosage form that can be administered orally. Pharmaceutical compositions can include one or more pharmaceutically acceptable excipients. Suitable excipients for solid dosage forms include sugars, starches, and other conventional substances including lactose, talc, sucrose, gelatin, carboxymethylcellulose, agar, mannitol, sorbitol, calcium phosphate, calcium carbonate, sodium carbonate, kaolin, alginic acid, acacia, corn starch, potato starch, sodium saccharin, magnesium carbonate, microcrystalline cellulose, colloidal silicon dioxide, croscarmellose sodium, talc, magnesium stearate, and stearic acid. Further, such solid dosage forms can be uncoated or can be coated to delay disintegration and absorption.

The pharmaceutical compositions can also be formulated for parenteral administration, e.g., formulated for injection via intravenous, intra-arterial, subcutaneous, rectal, subcutaneous, intramuscular, intraorbital, intracapsular, intraspinal, intraperitoneal, or intrasternal routes. Dosage forms suitable for parenteral administration include solutions, suspensions, dispersions, emulsions or any other dosage form that can be administered parenterally.

Pharmaceutically acceptable excipients are identified, for example, in The Handbook of Pharmaceutical Excipients, (American Pharmaceutical Association, Washington, D.C., and The Pharmaceutical Society of Great Britain, London, England, 1968). Additional excipients can be included in the pharmaceutical compositions of the invention for a variety of purposes. These excipients can impart properties which enhance retention of the compound at the site of administration, protect the stability of the composition, control the pH, facilitate processing of the compound into pharmaceutical compositions, and so on. Other excipients include, for example, fillers or diluents, surface active, wetting or emulsifying agents, preservatives, agents for adjusting pH or buffering agents, thickeners, colorants, dyes, flow aids, non-volatile silicones, adhesives, bulking agents, flavorings, sweeteners, adsorbents, binders, disintegrating agents, lubricants, coating agents, and antioxidants.

Some of the compounds and agents described herein can be prepared as a salt. For example, many of the vitamins that can comprise the “immune promoting agents” can be administered as acids (e.g., ascorbic acid) or a salt thereof (e.g., calcium ascorbate). “Salt” as used herein refers to pharmaceutically acceptable salts of the compounds described herein which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). Examples of pharmaceutically acceptable salts include, but are not limited to, nontoxic acid addition salts which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

In other embodiments, the compounds may be prepared as “prodrugs” in a pharmaceutically acceptable composition/formulation. As used herein, the term “prodrug” refers to a derivative of a compound that can hydrolyze, oxidize, or otherwise react under biological conditions (in vitro or in vivo) to provide a compound as described herein. Prodrugs may only become active upon some reaction under biological conditions, but they may have activity in their unreacted forms. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs and their uses are well known in the art (see, e.g., Berge, et al. 1977 J. Pharm. Sci. 66:1-19). Prodrugs can typically be prepared using well-known methods, such as those described in Burger's Medicinal Chemistry and Drug Discovery (1995, Manfred E. Wolff ed., 5thed. 172-178, 931-932).

As mentioned, the administration of the interferon or the extract containing the interferon, and an immune promoting agent and digestive enzyme according to the methods described herein can comprise the simultaneous or sequential administration of each component. Therefore, the interferon or an extract containing the interferon, immune promoting agent and digestive enzyme can be prepared as a single formulation or can be prepared in separate formulations. The term “combination” designates a treatment where at least two or more drugs are co-administered to a subject to cause a biological effect. In a combined therapy according to this invention, the at least two drugs may be administered together or separately, at the same time or sequentially. Also, the at least two drugs may be administered through different routes and protocols. As a result, although they may be formulated together, the drugs of a combination may also be formulated separately.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### 4. Additional Treatment Modalities and Combinations.

In accordance with the present invention, the treatment method described above may be combined with other established forms of treatment, like surgery, radiation therapy, cell therapy, or chemotherapy, or other treatments which support the function of the affected organ.

If the malignancy or pre-malignant condition affects the thyroid, the cell therapy can comprise thyroid cells obtained from a baby or fetal sheep. Also, in the case of an affected thyroid, percutaneous ethanol injections (PEI) may be administered at the pre-malignant stage in combination with an agent that promotes the function of an organ or organ system. For example, when a thyroid malignancy, pre-malignant condition or cancer is treated, the treatment can be administered alongside levothyroxine replacement therapy or other agents that promote thyroid function, with a daily dosage determined according to established procedures.

### 5. Conditions and Diseases to Treat

The methods described herein can comprise treating any malignancy, pre-malignancy, or cancer in a subject in need thereof. The cancer can be selected from the group consisting of thyroid, breast, ovarian, prostate, endometrial, colon, pancreatic, head and neck, gastric, renal, brain, liver, bladder, kidney, lung, esophageal, leukemia, multiple myeloma, lymphoma, and melanoma. For example, the malignancy and pre-malignant condition can be a condition of the thyroid or breast. Also, the pre-malignant condition can be selected from the group consisting of a typical ductal hyperplasia of the breast, actinic keratosis, leukoplakia, Barret's epithelium (columnar metaplasia) of the esophagus, ulcerative colitis, adenomatous colorectal polyps, erythroplasia of Queyrat, Bowen's disease, Bowenoid papulosis, vulvar intraepithelial neoplasia, dysplastic changes to the cervix, and dysplastic changes to the thyroid. Dysplastic changes to the thyroid can include but are not limited to: thyroiditis, microfollicular Hurthle cell patterns, trabecular groups, complex 3D groups, bubble gum colloids, giant cells, swirl pattherns, abnormal microfollicular patterns or any combination thereof. In addition, a pre-malignant or malignant condition of the thyroid can comprise the failure of a biopsied sample of an RNA translocation test, such as RET-PTC1, RET-PTC3, and PAX8-PPAR.

Preferably, the treatment comprising an interferon or an extract containing the interferon, immune promoting agent and digestive enzyme is administered to a subject afflicted with a pre-malignancy, malignancy or cancer of the thyroid. For example, the cancer can comprise a papillary thyroid carcinoma or medullary thyroid carcinoma.

In some cases, the malignancy, pre-malignant condition or cancer is unresponsive to traditional therapies. For example, the malignancy, pre-malignant condition or cancer may be unresponsive to chemotherapy, radiation therapy, or surgical resection. In addition, in rare cases, the malignancy, pre-malignant condition or cancer may be inoperable (e.g., localized to an area where surgical resection would result in unavoidable harm or death to the patient). Therefore, in rare cases, the methods of treating a malignancy, pre-malignancy, or cancer described herein can comprise administering an interferon or an extract comprising an interferon, an immune promoting agent and a digestive enzyme to a patient diagnosed with an inoperable tumor or cancer, or to a patient following exhaustion of traditional therapies (e.g., chemotherapy, radiation therapy, and/or surgery).

Excessive taurine and/or caffeine consumption may also lead to thyroid malignancies or pre-malignancies. Therefore, the subject may, prior to the administration of the treatment described herein, have consumed levels of taurine or caffeine higher than the recommended daily amount. In some cases, the subject may have consumed a daily amount of taurine greater than 80%, greater than 85%, greater than 90%, greater than 95% of the maximum daily recommended dose prior to diagnosis with the malignant or pre-malignant condition of the thyroid. In some cases, when the subject had consumed high levels of taurine and caffeine prior to diagnosis, the method further comprises limiting taurine and caffeine consumption to negligible levels. For example, the daily intake of taurine and caffeine during the treatment described herein can comprise less than 10%, less than 5%, or less than 3% of the daily recommended amount of taurine or caffeine. In some cases, the daily intake of taurine during treatment is less than 60 mg, 70 mg, 80 mg, 100 mg, 150 mg, 200 mg, 250 mg, 300 mg, 350 mg, 400 mg, 450 mg, or 500 mg. Preferably, the daily intake of taurine during treatment is negligible or less than 60 mg a day.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the invention in detail, it will be apparent that modifications and variations are possible without departing from the scope of the invention defined in the appended claims.

### Example

The following non-limiting example is provided to further illustrate the present invention.

The progression and treatment of a suspected papillary carcinoma of the thyroid is described in a 45 year old (80 kg) male patient.

Phase IA: Pre-Growth Period.

For about 48 months (4 years) prior to the presentation of any thyroid abnormalities, the patient consumed daily energy drinks providing a daily caffeine and taurine intake close to the maximum recommended daily allowance for each substance (400-500 mg caffeine and 3000 mg taurine).

Phase 1B: Tumor Presentation and Initial Treatment:

The patient presented with an enlarged left thyroid lobe, with liquid and solid components. Over a 42 month period the patient received levothyroxine replacement therapy and multiple percutaneous ethanol injections followed by targeted radiotherapy with 30 mCi ¹³¹I. Despite the standard treatment, the solid part of the cyst continued to grow. During the treatment, several biopsies were also obtained from the lower tip of the left lobe. Initially biopsies indicated a benign growth but by 42 months (about three and a half years) after initial presentation, biopsied cells had begun to show signs of secondary changes due to thyroid carcinoma. These included microfollicular Hurthle cell pattern, trabecular groups, complex 3D groups, bubble gum colloids, and giant cells, swirl patterns and abnormal microfollicular patterns. The biopsied cells also failed several RNA translocation tests, such as RET-PTC1, RET-PTC3, and PAX8-PPAR. Medullary thyroid carcinoma (MTC) and BRAF tests were negative. Self-palpitation also revealed the transition from benign growth to a cancerous tumor since over the same time the thyroid mass changed from a soft-tissue goiter to a hard mass characteristic of thyroid cancer. At its peak the left thyroid lobe measured 80 ml. During this initial treatment period, the patient also continued to consume levels of caffeine and taurine that approached the maximum recommended daily allowance (RDA) for each compound (400 mg-500 mg for caffeine and 3000 mg for taurine).

Phase II: Novel Therapy

The patient ceased caffeine and taurine consumption, limiting daily caffeine levels to less than 10 mg per day and keeping taurine consumption negligible. An intense enzyme supported immunotherapy was started combining natural interferons with immune boosters and digestive enzymes. The specific components taken are described in Table 1. Unless otherwise specified all components were taken daily for 20 months.

TABLE 1

| Component | Daily Dose | Administration frequency |
|---|---|---|
| Vitamin C (enteric coated) | 3000 mg | 1 x daily |
| Vitamin A | Unknown | 1 x daily |
| | (Generic product) | |
| Vitamin B6-B12 complex | Unknown | 1 x daily |
| | (Generic product) | |
| Echinacea | 600 mg | 2 x daily (300 mg each) |
| Ashwaghanda | 1200 mg | 4 x daily (300 mg each) |
| Kachnar bark extract | 600 mg | 2 x daily (300 mg each) |
| Serratiopeptidase | 360,000 active | 3 x daily (120,000 |
| | units (SPUs) | SPUs each) |

During the course of the treatment, self-palpitation of the goiter revealed both an apparent reduction in size as well as a favorable development regarding the “hardness” of the thyroid mass. Another biopsy obtained 13 months after initiation of the therapy revealed microfollicular groups and nuclear size variations, but did not display any of the other secondary changes typical for thyroid carcinoma that had been detected a year earlier. RNA translocation tests (RET-PTC1, RET-PTC3, and PAX8-PPAR) were negative.

A third biopsy was obtained 18 months after initiation of the novel therapy and both the ThyGenX Oncogene Classifier Status as well as the ThyraMIR microRNA Classifier Status were negative. These highly specific tests can exclude thyroid carcinoma at a 90% confidence level. A reduction in the total size of the left lobe down to about 20 ml, as estimated from ultrasound imaging, was also observed, with a concomitant softening of the entire lobe as determined by palpitation.

At 20 months after initiation of the novel therapy, baby or fetal sheep thyroid cells were injected into the patient to mediate regrowth of the thyroid.

Phase IV: Remission and Monitoring

Following the 20 month treatment protocol, the patient received modulating agents for thyroid function. These included kachnar (bauhinia tormentosa) (600 mg a day), thyroid SP-26 (a thyroid supplement that regulates ion intake and contains other herbal ingredients composed to support thyroid function) and reduced levothyroxine therapy. Further reduction of the goiter size, to about 13 ml, commensurate with a large, but not excessively large, thyroid lobe, was observed under ultrasound imaging, and thyroid function was observed to normalize. Daily caffeine consumption was gradually increased to a level of about 50 mg per day (about half a cup of coffee), while taurine consumption was negligible.

The treatment described above is summarized in Table 2 below.

TABLE 2

| | Months after | |
|---|---|---|
| Phase | initial diagnosis | Medical Report/Status/Treatment |
| Phase IA | −48-0 | Consumption of high amounts of caffeine and taurine. |
| Preclinical | | |
| Phase IB | 0 | Diagnosis of goiter; continued consumption of high amounts of caffeine |
| Diagnosis/ | | and taurine. |
| Initial Therapy | 1 | Draining of cyst and PEI, start of levothyroxine therapy (75 mcg a day); |
| | | biopsy indicates benign goiter; continued consumption of high amounts |
| | | of caffeine and taurine. |
| | 19 | Reduction of liquid part of cyst noted but continued growth of solid |
| | | portion; second percutaneous PEI; second biopsy indicates benign |
| | | goiter; continued consumption of high amounts of caffeine and taurine; |
| | | continued levothyroxine therapy (75 mcg a day). |
| | 27 | Liquid part of cyst has disappeared but continued growth of solid |
| | | portion; radioactive therapy with ¹³¹I (30 mCi source) to reduce solid |
| | | part of goiter; energy drink consumption ceases (no caffeine and |
| | | taurine intake); continued levothyroxine therapy (75 mcg a day). |
| | 43 | No effect from radiotherapy, solid portion of goiter grows and |
| | | palpitation reveals firm and solid areas within goiter mass and biopsy |
| | | shows suspicious areas indicative of thyroid carcinoma; solid portion of |
| | | goiter measures at 80 ml; no caffeine and taurine consumption; |
| | | continued levothyroxine therapy (75 mcg a day). |
| Phase II - | 43-63 | Initiate daily immunotherapy treatment of vitamin C (3000 mg), vitamins |
| Immunotherapy | | A and B6-B12, Echinacea (600 mg), Ashwaghanda extract (1200 mg), |
| | | kachnar bark extract (600 mg), serratiopeptidase (360,000 SPUs); |
| | | continued levothyroxine therapy (75 mcg a day); no caffeine and |
| | | taurine consumption. |
| | 56 | Biopsy revealed microfollicular groups and nuclear size variations, but |
| | | none of the other secondary changes typical for thyroid carcinoma |
| | | detected before. RNA translocation tests (RET-PTC1, RET-PTC3, and |
| | | PAX8-PPAR) were negative. |
| | 61 | Biopsy. Both ThyGenX Oncogene Classifier Status as well as |
| | | ThyraMIR microRNA Classifier Status were negative. Size of lobe was |
| | | 20 ml and was soft, based on palpitation. |
| | 63 | Cell therapy. Fetal or baby sheep thyroid cells were injected into |
| | | patient. |
| Phase III - | 63-79 | Reduced levothyroxine therapy; modulating agents for thyroid function |
| Remission/ | | administered (kachnar and thyroid SP-26); further reduction in goiter |
| Recovery | | size to about 13 ml observed, thyroid function normalized. Daily |
| | | caffeine consumption increased to a level of about 50 mg per day while |
| | | taurine consumption was maintained at zero level. |

### Definitions

Definitions described herein are provided to better define the present disclosure and to guide those of ordinary skill in the art in the practice of the present disclosure. Unless otherwise noted, terms are to be understood according to conventional usage by those of ordinary skill in the relevant art.

Numbers expressing quantities of ingredients, properties such as molecular weight, reaction conditions, and so forth, used to describe and claim certain embodiments of the present disclosure are to be understood as being modified in some instances by the term “about.” The term “about” is used to indicate that a value includes the standard deviation of the mean for the device or method being employed to determine the value. The numerical parameters set forth in the written description and attached claims are approximations that can vary depending upon the desired properties sought to be obtained by a particular embodiment of the present disclosure. The numerical parameters should be construed in light of the number of reported significant digits and by applying ordinary rounding techniques. Notwithstanding that the numerical ranges and parameters setting forth the broad scope of the present disclosure are approximations, the numerical values set forth in the specific examples are reported as precisely as practicable. The numerical values presented in the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

The term “or” as used herein, including the claims, is used to mean “and/or” unless explicitly indicated to refer to alternatives only or the alternatives are mutually exclusive.

All methods described herein can be performed in any suitable order unless otherwise indicated herein or otherwise clearly contradicted by context. The use of any and all examples, or exemplary language (e.g. “such as”) provided herein is intended merely to better illuminate the present disclosure and does not pose a limitation on the scope of the disclosure otherwise claimed. No language in the specification should be construed as indicating any non-claimed element essential to the practice of the invention.

Groupings of alternative elements disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all groups used in the appended claims.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there can be additional elements other than the listed elements.

In view of the above, it will be seen that the several objects of the invention are achieved and other advantageous results attained.

As various changes could be made in the above methods without departing from the scope of the invention, it is intended that all matter contained in the above description shall be interpreted as illustrative and not in a limiting sense.

1. A method for treating a malignancy, a pre-malignant condition or cancer comprising administering an interferon or an extract containing an interferon, an immune promoting agent, and a digestive enzyme to a subject in need thereof.

2. (canceled)

3. The method of claim 1 wherein the extract containing an interferon comprises an extract of withaferania somnia or Ashwaghanda.

4. The method of claim 1 wherein the interferon comprises withaferin A.

5. The method of claim 1 wherein the extract containing the interferon is administered at a dose of about 500 mg to about 2000 mg, from about 600 mg to about 1800 mg, from about 800 mg to about 1600 mg, or from about 1000 mg to about 1400 mg a day.

6. The method of claim 1 wherein the immune promoting agent comprises a vitamin.

7. The method of claim 6 wherein the vitamin comprises Vitamin C, Vitamin A Vitamin B₆, Vitamin B₁₂ or any combination thereof.

8. The method of claim 7 wherein the vitamin comprises Vitamin C.

9. The method of claim 1 wherein the immune promoting agent is administered at a dose of about 30 to about 40 times the recommended daily amount (RDA) for the agent.

10. The method of claim 1 wherein the immune promoting agent comprises Vitamin C and is administered at a dose of at least 2000 mg per day or about 3000 mg per day.

11. The method of claim 1 wherein the digestive enzyme comprises serratiopeptidase or serrapeptase.

12. The method of claim 1 wherein the digestive enzyme is administered at a dose of about 300,000 to about 400,000 active units, from about 310,000 to about 390,000 active units, or from about 330,000 to about 370,000 active units per day.

13. The method of claim 1 wherein the malignancy, pre-malignant condition, or cancer comprises a thyroid, breast, ovarian, prostate, endometrial, colon, pancreatic, head and neck, gastric, renal, brain, liver, bladder, kidney, lung, esophageal, leukemia, multiple myeloma, lymphoma, or melanoma malignancy or cancer.

14. The method of claim 13 wherein the cancer comprises a papillary carcinoma of the thyroid.

15. The method of claim 1 further comprising administering at least one herbal supplement.

16. The method of claim 15 wherein the herbal supplement comprises Echinacea or kachnar (Bauhinia tormentosa) bark extract.

17. The method of claim 1 further comprising administering a thyroid modulating agent.

18. The method of claim 17 wherein the thyroid modulating agent comprises levothyroxine.

19. The method of claim 1 wherein the subject in need thereof is unresponsive to chemotherapy or radiation therapy and/or the subject is ineligible for surgery and resection of the cancer or the malignancy.

20. The method of claim 1 wherein taurine intake by the subject during treatment is less than 60, 70, 80, 10, 150, 200, 250, 300, 350, 400, 450, or 500 mg per day.

21. A method for treating a malignancy, a pre-malignant condition or cancer comprised of a papillary carcinoma of the thyroid, the method comprising administering to a subject in need thereof:
an interferon containing withaferin A or an extract containing withaferania somnia or Ashwaghanda at a daily dose of about 500 mg to about 2000 mg,
an immune promoting agent comprising Vitamin C, Vitamin A, Vitamin B₆, Vitamin B₁₂ or any combination thereof at a daily dose of about 30 to about 40 times the recommended daily amount (RDA) for the immune promoting agent, and
a digestive enzyme containing serratiopeptidase or serrapeptase at a daily dose of bout 300,000 to, about 400,000 active units.
